(19) United States
(12) Patent Application Publication
(10) Pub. No.: US 2024/0415778 A1
(43) Pub. Date: Dec. 19, 2024

(54) ORAL PREPARATION CONTAINING PROGESTOGEN, AND PREPARATION METHOD AND USE

(71) Applicant: SHENZHEN EVERGREEN THERAPEUTICS CO., LTD., Shenzhen, Guangdong (CN)

(72) Inventors: Xin DU, Shenzhen City (CN); Shanshan LI, Shenzhen City (CN); Tao Tom DU, Shenzhen City (CN)

(73) Assignee: SHENZHEN EVERGREEN THERAPEUTICS CO., LTD., Shenzhen, Guangdong (CN)

(21) Appl. No.: 18/706,344

(22) PCT Filed: Oct. 17, 2022
(86) PCT No.: PCT/CN2022/125665

§ 371 Date: Apr. 30, 2024

(30) Foreign Application Priority Data
Nov. 5, 2021 (CN) 202111306285.8

(51) Int. Cl. A61K 9/48; A61K 9/00; A61K 9/08; A61K 31/57; A61K 47/14; A61K 47/34

(52) U.S. Cl. CPC A61K 9/4866; A61K 9/0053; A61K 9/08; A61K 31/57; A61K 47/14; A61K 47/34

(57) ABSTRACT

An oral preparation containing a progestogen, and a preparation method and the use. The oral preparation does not contain or contains only benzyl benzoate in a weight percentage of less than 8%. The preparation contains 17-α hydroxyprogesterone caproate, propylene glycol monocaprylate, PEG35 castor oil, and propylene glycol monolaurate. The oral preparation provides good stability, dissolution performance, and bioavailability while ensuring high safety.

### CROSS REFERENCE TO RELATED APPLICATIONS

This application claims priority to the Chinese patent application filed with the State Intellectual Property Office on Nov. 5, 2021, under application No. 202111306285.8, titled “Novel Oral Preparation Containing Progestogen and Preparation Method and Use” The entire content of said Chinese patent application is incorporated herein by reference.

### TECHNICAL FIELD

This application belongs to the field of pharmaceuticals. Specifically, the application provides a novel oral formulation containing reproductive hormones, along with its preparation method and applications. The formulation does not contain or contains only low levels of benzyl benzoate.

### BACKGROUND

Hormones, including progesterone, levonorgestrel, norethindrone, 17-α hydroxyprogesterone, among others, exhibit diverse physiological functions in the human body. Currently, 17-α hydroxyprogesterone is employed for contraception and the treatment of conditions such as miscarriage, menstrual irregularities, uterine bleeding, and breast cancer. Moreover, the applicant has discovered the therapeutic effect of 17-α hydroxyprogesterone in treating cytokine storm syndrome and has filed a patent application for this purpose.

Oral formulations are significantly more convenient and compliant compared to other dosage forms such as injections. The applicant has previously collaborated with other entities to develop an oral formulation of 17-α hydroxyprogesterone and obtained a patent for it. This formulation demonstrates excellent oral bioavailability and stability. However, the primary solvent component in this formulation is benzyl benzoate, and the safety of benzyl benzoate for human consumption has not been clearly established. According to the regulations of the Joint FAO/WHO Expert Committee on Food Additives (JECFA), the acceptable daily intake (ADI) of benzyl benzoate, expressed as benzoic acid equivalent, is limited to 5 mg/kg.

These issues pose obstacles to the broader practical application of the existing oral formulation of 17-α hydroxyprogesterone, and thus, there is a compelling need to develop a safer 17-α hydroxyprogesterone oral formulation with low or no benzyl benzoate.

### SUMMARY OF THE INVENTION

To address the aforementioned issues, the applicant conducted extensive screening of excipients based on their fundamental properties and safety. They designed an oral formulation with high safety, excellent stability, dissolution performance, and bioavailability.

On one hand, this application provides an oral formulation containing hormones, characterized by the absence or low levels of benzyl benzoate.

Further, the hormone is 17-α hydroxyprogesterone.

Further, the weight percentage of benzyl benzoate in the oral formulation is below 8%, preferably below 7%, more preferably below 6%, and even more preferably below 5%.

Further, the weight percentage of benzyl benzoate in the oral formulation is below 4%, preferably below 3%, more preferably below 2%, and even more preferably below 1%.

Further, the oral formulation does not contain benzyl benzoate.

Further, the oral formulation includes propylene glycol monocaprylate, preferably Capryol®90.

Further, the weight percentage of propylene glycol monocaprylate in the oral formulation is between 40-60%, preferably 45-55%, more preferably 50-52%, and even more preferably 51.85%.

Further, the formulation also contains PEG35 castor oil, preferably Kolliphor®ELP.

Further, the weight percentage of PEG35 castor oil in the oral formulation is between 5-10%, preferably 6-9%, more preferably 7-8%, and even more preferably 7.65%.

Further, the weight percentage of progesterone in the oral formulation is between 10-20%, preferably 12-18%, more preferably 15-17.5%.

Further, the formulation further includes propylene glycol monolaurate, preferably Lauroglycol®90.

Further, the weight percentage of propylene glycol monolaurate in the oral formulation is between 15-30%, preferably 20-25%, more preferably 20.8%-20.9%, and most preferably 20.83%.

Further, the oral formulation contains glyceryl monooctanoate, preferably Capmul 808G.

Further, the weight percentage of glyceryl monooctanoate in the oral formulation is between 5-25%, preferably 8-22%.

Further, the oral formulation comprises 17-α-hydroxyprogesterone, propylene glycol monocaprylate, PEG35 castor oil, benzyl benzoate, and either propylene glycol monolaurate or glyceryl monooctanoate.

Further, the oral formulation consists of the following weight percentages: 10-20% 17-α-hydroxyprogesterone, 45-55% propylene glycol monocaprylate, 5-10% PEG35 castor oil, 1-6% benzyl benzoate, and 15-25% propylene glycol monolaurate or 9-22% glyceryl monooctanoate.

Further, the oral formulation is composed of 15% 17-α-hydroxyprogesterone, 51.8%-51.9% propylene glycol monocaprylate (preferably 51.85% propylene glycol monocaprylate), 7.65% PEG35 castor oil, 4.6-4.7% benzyl benzoate (preferably 4.675% benzyl benzoate), and 20.8-20.9% propylene glycol monolaurate (preferably 20.825% propylene glycol monolaurate).

Or it consists of 17.5% 17-α-hydroxyprogesterone, 60.64% propylene glycol monocaprylate, 7.43% PEG35 castor oil, 5.355% benzyl benzoate, and 9.075% glyceryl monooctanoate.

Or it can be composed of 15% 17-α-hydroxyprogesterone, 51.8%-51.9% propylene glycol monocaprylate (preferably 51.85% propylene glycol monocaprylate), 7.65% PEG35 castor oil, 4.6-47% benzyl benzoate (preferably 4.675% benzyl benzoate), and 20.8-20.9% glyceryl monooctanoate (preferably 20.825% glyceryl monooctanoate).

Further, the oral formulation comprises 17-α-hydroxyprogesterone, propylene glycol monocaprylate, PEG35 castor oil, and propylene glycol monolaurate.

Further, the oral formulation contains the following weight percentages: 10-20% 17-α-hydroxyprogesterone, 45-55% propylene glycol monocaprylate, 5-10% PEG35 castor oil, and 20-30% propylene glycol monolaurate.

Further, the oral formulation is composed of 15% 17-α-hydroxyprogesterone, 51.85% propylene glycol monocaprylate, 7.65% PEG35 castor oil, and 25.50% propylene glycol monolaurate.

Further, the oral formulation of the present application is in the form of oral solution or soft capsule, with a preference for the soft capsule form.

Further, each dosage unit contains 80-150 mg of the hormone, preferably 100-120 mg.

Further, the oral formulation is intended for contraception or for the treatment of miscarriage, menstrual irregularities, uterine bleeding, breast cancer, cytokine storm syndrome, or COVID-19.

On the other hand, the present application provides a method for contraception or the treatment of miscarriage, menstrual irregularities, uterine bleeding, breast cancer, cytokine storm syndrome, or COVID-19, comprising the step of using the aforementioned oral formulation.

In this application, 17-α-hydroxyprogesterone is also referred to as hydroxyprogesterone caproate, 17-HPC, 17-caproxyprogesterone, 17-hydroxycapronate, among other names. Its CAS number is 630-56-8, and in certain embodiments and figures of the application, it is abbreviated as HPC. The term “low levels of benzyl benzoate” in this application refers to the intake level of benzyl benzoate (or benzyl benzoate equivalent) when the formulation of the present application is taken in accordance with the dosage, complying with the recommended/required levels of various countries, organizations, or textbooks. Preferably, the intake level of benzyl benzoate (or benzyl benzoate equivalent) when the formulation of the present application is taken at the recommended dosage is significantly lower than the recommended/required levels of various countries, organizations, or textbooks.

The mentioned trade names such as Capryol®90, Lauroglycol®90, Capmul 808G, Peceol®, Maisine®CC, Koliphor®HS15, Koliphor®ELP, Koliphor®RH40, and others in this application do not imply that the protection scope is limited to those specific products. Similar or identical products with the same chemical composition and physical properties are also within the protection scope of this application. As a non-limiting example, Capryol®90 used in the examples of this application, other propylene glycol monocaprylate products may be chosen by those skilled in the art based on their needs and performance.

In addition to the aforementioned oral solution and soft capsule formulations, other oral dosage forms containing the liquid formulation provided by this application are also within the protection scope of this application.

In addition to the aforementioned indications, other diseases known or under research that can be treated with progesterone are also within the pharmaceutical utility protection scope of this application.

Depending on the therapeutic needs, the combination in this application does not exclude the inclusion of other known and unknown drugs for treating related diseases, or these drugs can be co-administered with the combination in this application. These drugs include but are not limited to anti-inflammatory drugs, antipyretic drugs, antitumor drugs, immunomodulatory drugs, and the like.

This application improves the chemical stability of the oral formulation with low benzyl benzoate, enhancing its clinical and commercial utility and value. High chemical stability of the formulation can reduce production costs, as well as storage and transportation costs, thereby increasing the economic and commercial value of the product.

The application enhances the bioavailability of the oral formulation with low benzyl benzoate, which can reduce the dosage of the drug and improve the practicality of its use by patients. Experimental results show that the dissolution rate of the oral formulation with low benzyl benzoate in simulated intestinal fluid during fasting is over 8.8 times higher than that of the original formulation with high benzyl benzoate. This demonstrates that the bioavailability of the low benzyl benzoate formulation is more than 8.8 times higher than that of the original high benzyl benzoate formulation, allowing for a significantly lower dosage compared to the original high benzyl benzoate formulation. This significantly increases the clinical and commercial application value of the low benzyl benzoate formulation.

The safety of the low benzyl benzoate formulation in this application is greatly improved compared to the original high benzyl benzoate formulation. The excipients used in the low benzyl benzoate formulation comply with safety requirements, and the daily amount of excipients used also meets safety standards and requirements. This significantly expands the range of use and commercial value of the low benzyl benzoate formulation.

### BRIEF DESCRIPTION OF THE DRAWINGS

FIG. 1 depicts the physical images of various formulations during the 72-hour stability experiment at 5° C.

FIG. 2 presents the physical images of different formulations during the 72-hour stability experiment at 12.5° C.

FIG. 3 illustrates the physical images of various formulations during the 72-hour stability experiment at 20° C.

FIG. 4 shows the dissolution performance of formulations 210106, 210112, and BB in FaSSIF (fasted-state simulated intestinal fluid) through complete sample analysis.

FIG. 5 displays the dissolution performance of formulations 210106, 210112, and BB in FaSSIF through water-phase sample analysis.

FIG. 6 exhibits the physical photographs of solvent samples in FaSSGF (fasted-state simulated gastric fluid) after 120 minutes of dissolution stirring.

FIG. 7 illustrates the dissolution performance of formulations 210106, 210112, and BB in FaSSGF through complete sample analysis.

FIG. 8 demonstrates the dissolution performance of formulations 210106, 210112, and BB in FaSSGF through water-phase sample analysis.

FIG. 9 presents the physical photographs of solvent samples in FeSSGF (fed-state simulated gastric fluid) after 120 minutes of dissolution stirring.

FIG. 10 showcases the dissolution performance of formulations 210106, 210112, and BB in FeSSIF (fed-state simulated intestinal fluid) through complete sample analysis.

FIG. 11 displays the dissolution performance of formulations 210106, 210112, and BB in FeSSIF through water-phase sample analysis.

FIG. 12 compares the dissolution performance of 210106 in FeSSIF, FaSSIF, and FaSSGF.

FIG. 13 compares the dissolution performance of 210112 in FeSSIF, FaSSIF, and FaSSGF.

FIG. 14 compares the dissolution performance of the BB formulation in FeSSIF, FaSSIF, and FaSSGF.

### DETAILED DESCRIPTION

### Example 1 Formulation Determination

Through screening based on fundamental physical properties, toxicity, and relevant standards, we have selected 10 excipients for formulating the recipe. These include Propylene Glycol Monocaprylate (Capryol®90), Propylene Glycol Monolaurate (Lauroglycol®90), Mono-Octanoin Glyceride (Capmul 808G), Oleic Acid Glyceride (Peceol®), Glycerol Monooleate (Maisine®CC), Oleic Acid, Olive Oil, PEG15 Hydroxystearate (Koliphor®HS15), PEG35 Castor Oil (Kolliphor®ELP), and PEG40 Hydrogenated Castor Oil (Koliphor®RH40). These excipients can be obtained using the product names listed in Table 1, such as Capryol®90, Lauroglycol®90, Capmul 808G, Peceol®, Maisine®CC, Oleic Acid, Olive Oil, Koliphor®HS15, Kolliphor®ELP, and Koliphor®RH40. Alternatively, other brands of these products can also be used. The mentioned product names are provided for clarity and are not intended to limit the scope of this application.

The solubility performance of these excipients was initially investigated, and the basic results are presented in Table 1:

TABLE 1
Solubility Property of 17-HPC in Different Excipients

| | | | | | | Suggested working |
|---|---|---|---|---|---|---|
| | | | | [symbol] ment Condition | | concentrations based on |
| | | | [symbol] ed | Dissolving | Dissolving | physical stability and |
| | [symbol] C Excipient | | [symbol] bunds | Temperature | Time | saturated solubility |
| | Chemical Name | Brand Name | [symbol] tration | (° C.) | (min) | results (mg/g) |
| 1 | Propylene | Capryol ® 90 | >150 mg/g | 30° C. | 30 | 150 |
| | Glycol | | | | | |
| | Monocaprylate | | | | | |
| 2 | Propylene | Lauroglycol ® | >100 mg/g | 30° C. | 45 | 100 |
| | Glycol | 90 | | | | |
| | Monolaurate | | | | | |
| 3 | Mono-Octanoin | Capmul 808G | >100 mg/g | 50° C. | 45 | 120 |
| | Glyceride | | | | | |
| 4 | Oleic Acid | Peceol ® | >50 mg/g | 50° C. | 45 | 50 |
| | Glyceride | | | | | |
| 5 | Glycerol | Maisine ® CC | >50 mg/g | 30° C. | 90 | 65 |
| | Monooleate | | | | | |
| 6 | Oleic Acid | — | >50 mg/g | 30° C. | 60 | 70 |
| 7 | Olive Oil | — | >12.5 mg/g | 30° C. | 90 | Not effective for adult |
| | | | | | | formulations |
| 8 | PEG15 | Kolliphor ® | >50 mg/g | 50° C. | 45 | Chemically unstable |
| | Hydroxystearate | HS15 | | | | |
| 9 | PEG35 Castor | Kolliphor ® | >25 mg/g | 50° C. | 45 | 30 |
| | Oil | ELP | | | | |
| 10 | PEG40 | Kolliphor ® | >25 mg/g | 50° C. | 60 | Chemically unstable |
| | Hydrogenated | RH40 | | | | |
| | Castor Oil | | | | | |

[symbol]

indicates data missing or illegible when filed

The data in Table 2 indicates that 17-HPC achieves stable solubility in excipients such as propylene glycol monocaprylate, propylene glycol monolaurate, caprylic/capric triglyceride, glyceryl caprylate, glyceryl oleate, oleic acid, and PEG35 castor oil. However, the stable solubility concentrations vary for each excipient, with specific values provided in the last column of Table 2. This study reveals that 17-HPC cannot achieve stable solubility in PEG15 hydroxystearate and PEG40 hydrogenated castor oil. The stable solubility concentration of 17-HPC in olive oil is insufficient to meet the meaningful solubility concentration required for adult formulations.

Based on the WHO-JECFA database or acceptable daily intake values provided by suppliers, and considering the potential API dosage and formulation dosage, formulations 210104, 210106, 210108, 210110, 210112, and 210114 were designed:

TABLE 2
Basic Information of Excipients

| | | Acceptable Daily | |
|---|---|---|---|
| Chemical Name of | | Intake (ADI) | ADI/Weight |
| Excipients | Brand Name | (mg/kg/day) | 60 kg |
| Propylene glycol | Capryol ® 90 | 56 | 3.36 |
| monocaprylate | | | |
| Propylene glycol | Lauroglycol 90 | 83 | 4.98 |
| monolaurate | | | |
| Glycerol | Capmul 808G | none | none |
| monocaprylate | | | |
| PEG35 castor oil | Kolliphor ® ELP | 25 | 1.50 |
| Benzyl benzoate | — | 5 | 0.30 |

TABLE 3
Oral Formulation Containing 17-HPC

| | | | | | Excipient | | | | | | |
|---|---|---|---|---|---|---|---|---|---|---|---|
| | | | | | Mixture | Excipient | | | | | |
| | | | | Dosage of | Amount | Amount | | | | | |
| | | | | Formulation | for | for | ADI | | | | |
| | | | API | containing | Dosage | Dosage | Value | | | Formulation | Formulation |
| Formu- | Chemical | | (17-HPC) | 960 mg | of | of | for | | Disperse | Physical | Physical |
| lation | Name of | Ratio | Concen- | 17-HPC. | 960 mg | 960 mg | 60 kg | Dis- | in Water | Stability | Stability |
| # | Excipient | (wt. %) | tration | (g) | HP (g) | HPC (g) | Adult | solving | at 37° C. | (one week) | (two weeks) |
| Formula | Capryol ® | 73.5 | 175 | 5.486 | 4.526 | 3.327 | √ | √ | No API | Stable | Crystal- |
| 1 | 90 | | mg/g | | | | | | pre- | at room | lization |
| 210104 | Kolliphor ® | 9 | | | | 0.407 | √ | | cipitation | temperature | observed |
| | ELP | | | | | | | | (only 1-2 | after one | after two |
| | Benzyl | 6.5 | | | | 0.294 | √ | | small | week | weeks |
| | benzoate | | | | | | | | crystals | | (13 days) |
| | Lauroglycol ® | 11 | | | | 0.498 | √ | | observed | | |
| | 90 | | | | | | | | from | | |
| | | | | | | | | | T1H | | |
| | | | | | | | | | onwards) | | |
| Formula | Capryol ® | 61 | 150 | 6.4 | 5.44 | 3.318 | √ | √ | No API | Stable | Stable |
| 2 | 90 | | mg/g | | | | | | pre- | at room | at room |
| 210106 | Kolliphor ® | 9 | | | | 0.490 | √ | | cipitation | temperature | temperature |
| | ELP | | | | | | | | in 24 H | after one | after two |
| | Benzyl | 5.5 | | | | 0.299 | √ | | | week | weeks |
| | benzoate | | | | | | | | | | (13 days) |
| | Lauroglycol ® | 24.5 | | | | 1.333 | √ | | | | |
| | 90 | | | | | | | | | | |
| Formula | Capryol ® | 73.5 | 175 | 5.486 | 4.526 | 3.327 | √ | √ | No API | Stable | Stable |
| 3 | 90 | | mg/g | | | | | | pre- | at room | at room |
| 210108 | Kolliphor ® | 9 | | | | 0.407 | √ | | cipitation | temperature | temperature |
| | ELP | | | | | | | | in 24 H | after one | after two |
| | Benzyl | 6.5 | | | | 0.294 | √ | | | week | weeks |
| | benzoate | | | | | | | | | | (13 days) |
| | Capmul | 11 | | | | 0.498 | √ | | | | |
| | 808G | | | | | | | | | | |
| Formula | Capryol@ | 61 | 150 | 6.4 | 5.44 | 3.318 | √ | √ | No API | Stable | Stable |
| 4 | 90 | | mg/g | | | | | | pre- | at room | at room |
| 210110 | Kolliphor ® | 9 | | | | 0.490 | √ | | cipitation | temperature | temperature |
| | ELP | | | | | | | | in 24 H | after one | after two |
| | Benzyl | 5.5 | | | | 0.299 | √ | | | week | weeks |
| | benzoate | | | | | | | | | | (13 days) |
| | Capmul | 24.5 | | | | 1.333 | √ | | | | |
| | 808G | | | | | | | | | | |
| Formula | Capryol ® | 61 | 150 | 6.4 | 5.44 | 3.318 | √ | √ | No API | Stable | Stable |
| 5 | 90 | | mg/g | | | | | | pre- | at room | at room |
| 210112 | Kolliphor ® | 9 | | | | 0.490 | √ | | cipitation | temperature | temperature |
| | ELP | | | | | | | | in 24 H | after one | after two |
| | Lauroglycol ® | 30 | | | | 1.632 | √ | | | week | weeks |
| | 90 | | | | | | | | | | (13 days) |
| Formula | Capryol ® | 61 | 150 | 6.4 | 5.44 | 3.318 | √ | √ | API | Detection | Detection |
| 6 | 90 | | mg/g | | | | | | crystal- | stopped | stopped |
| 210114 | Kolliphor ® | 9 | | | | 0.490 | √ | | lization | | |
| | ELP | | | | | | | | observed | | |
| | Capmul | 30 | | | | 1.632 | √ | | at | | |
| | 808G | | | | | | | | 3 hours | | |

Note:
ADI means allowable daily intake, √ means compliant.

The above table indicates that the selected six formulations are compliant with the safe intake levels. Experimental results show that, except for formulations 1 and 6, the remaining four formulations maintain physical stability when dispersed in the aqueous phase without crystal precipitation. Therefore, further research will be conducted on these four formulations.

The above formulations were screened, and the preferred formulations were 210106, 210108, 210110, and 210112.

TABLE 4
The Selected Preferred Oral Formulation including 17-HPC

Formulation #

| | 210106 | 201108 | 210110 | 210112 |
|---|---|---|---|---|
| Component | Wt. % | Wt. % | Wt. % | Wt. % |
| Capryol ®90 | 51.85 | 60.64 | 51.85 | 51.85 |
| Koliphor ®ELP | 7.65 | 7.43 | 7.65 | 7.65 |
| Benzyl benzoate | 4.675 | 5.355 | 4.675 | — |
| Lauroglycol ®90 | 20.825 | — | — | 25.50 |
| Capmul 808G | — | 9.075 | 20.825 | — |
| 17-HPC | 15.00 | 17.50 | 15.00 | 15.00 |
| Total (g) | 100 | 100 | 100 | 100 |

### Example 2 Basic Preparation Process of Formulation

Prepare 50 g of each formulation using the following steps:

A. Weigh the required excipients in a 100 ml bottle (pre-dissolved).

B. Mix the excipients under magnetic stirring (approximately 250 RPM) for 5 minutes.

C. Add the weighed API (17-HPC).

D. Stir magnetically at room temperature (250 RPM) for 1 hour; then at 30° C. in a water bath (250 RPM) for 3 hours.

E. After dissolution is complete, perform tests to ensure complete dissolution.

The preparation method for capsule formulation 210106 is as follows:

### Step 1—Formulation Preparation

The container containing Kolliphor® ELP needs to be preheated to 50° C.±5° C. until completely melted and homogenized before weighing.

All steps of formulation preparation are carried out under nitrogen purging.

In a container, mix the required quantities of Capryol®90 (2.593 kg), Lauroglycol®90 (1.041 kg), and benzyl benzoate (0.234 kg) until homogenized at room temperature.

Add the required amount of 17-HPC active pharmaceutical ingredient (0.75 kg) and stir thoroughly at 30° C.±5° C. until completely dissolved to obtain a clear solution without any suspended API particles.

The required quantity of pre-melted Kolliphor® ELP (0.382 kg) will be added to the container, and the complete formulation will be mixed thoroughly at 30° C.±5° C. until fully dissolved.

The formulation stored in sealed containers will be gently stirred at 25° C.±5° C. until completely degassed (i.e., a clear homogeneous solution without bubbles). The formulation will be stored at room temperature until packaging.

TABLE 5
Capsule Formulation Ingredients Table

| | | | | Weighted | |
|---|---|---|---|---|---|
| | | Amount | | Quantity | |
| Ingredient | Function | %(w/w) | (mg/capsule) | Batch | |
| Filling Component (Active Formula) | | | | 5 kg | |
| 17-HPC | Active | 15.00 | 100.00 | 0.75 | kg |
| | Ingredient | | | | |
| Propylene glycol | Solubilizer | 51.850 | 345.67 | 2.593 | kg |
| monocaprylate | | | | | |
| (Capryol ® 90) | | | | | |
| Propylene glycol | Solubilizer | 20.825 | 138.83 | 1.041 | kg |
| monolaurate | | | | | |
| (Lauroglycol ® 90) | | | | | |
| Macrogolglycerol | Surfactant | 7.650 | 51.00 | 0.382 | kg |
| ricinoleate | | | | | |
| (Kolliphor ® ELP) | | | | | |
| Benzyl benzoate | Solubilizer | 4.675 | 31.17 | 0.234 | kg |

| | | | | |
|---|---|---|---|---|
| Total filling formulation | 100.000 | 666.67 | 5.000 | kg |

### Example 3 Physical Stability at Different Temperatures

The stability of the preferred formulation in 72 hours at different temperatures was observed, with the results shown in Table 5.

The stability of the preferred formulation at different temperatures in different time periods was observed, with the results shown in Table 6.

TABLE 6
Observation Result of Different Formulation in Different Time Periods
(3 Days, 2 weeks and 1 month)

| Temperature | 5° C. | | | 12.5° C. | | | ~20° C. | | |
|---|---|---|---|---|---|---|---|---|---|
| Formulation | 3 days | 2 weeks | 1 month | 3 days | 2 weeks | 1 month | 3 days | 2 weeks | 1 month |
| 210106 | Stable | Less | Unstable | Stable | Stable | Stable | Stable | Stable | Stable |
| | | stable | | | | | | | |
| 210108 | Unstable | Unstable | Unstable | Unstable | Unstable | Unstable | Less | Less | Less |
| | | | | | | | stable | stable | stable |
| 210110 | Unstable | Unstable | Unstable | Unstable | Unstable | Unstable | Stable | Stable | Stable |
| 210112 | Less | Unstable | Unstable | Less | Less | Unstable | Less | Less | Less |
| | stable | | | stable | stable | | stable | stable | stable |

Note:

Stable means no crystalline deposits appear;

Less stable means a small amount of deposit appear, such as 1-3 crystalline deposits;

Unstable means a large amount of crystalline deposit appear.

The results indicate that all formulations are sensitive to low temperatures (5° C.), with 210106 being the only formulation that remains stable for one month at 12.5° C. and 20° C.

The stability assessment for 210106 over longer periods (3 days, 2 weeks, 1 month, 2 months, 3 months, 4 months) is shown in Table 7.

TABLE 7
Observation Result of 210106 in Long Time Test
(3 Days, 2 Weeks, 1 Month, 2 Months, 3 Months, 4 Months)

| | Time | | | | | |
|---|---|---|---|---|---|---|
| | 3 days | 2 weeks | 1 month | 2 months | 3 months | 4 months |
| | Temperature 5° C. | | | | | |
| Stability | Stable | Less stable | Unstable | Unstable | Unstable | Unstable |
| | Temperature 12.5° C. | | | | | |
| Stability | Stable | Stable | Stable | Stable | Stable | Stable |
| | Temperature ~20° C. | | | | | |
| Stability | Stable | Stable | Stable | Stable | Stable | Stable |

Note:

Stable means no crystalline deposits appear;
Less stable means a small amount of deposit appear, such as 1-3 crystalline deposits;
Unstable means a large amount of crystalline deposit appear.

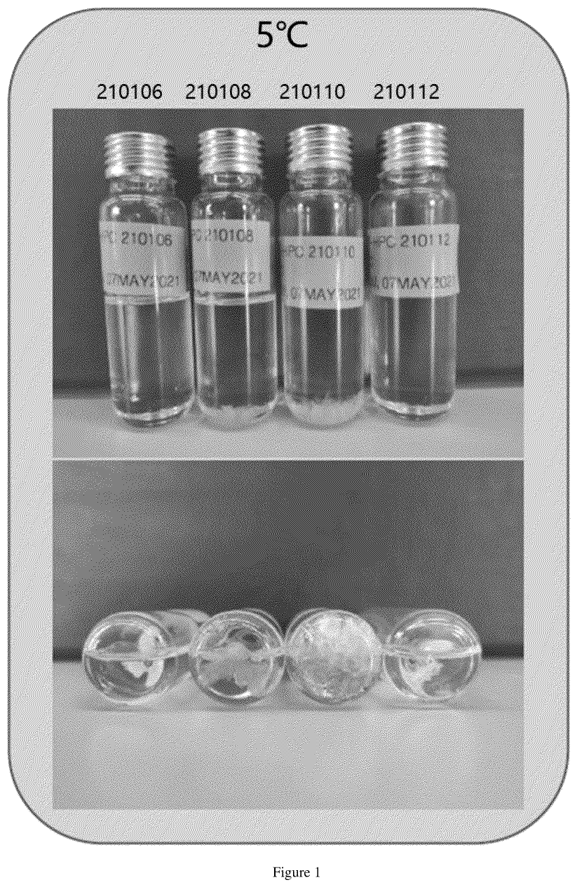

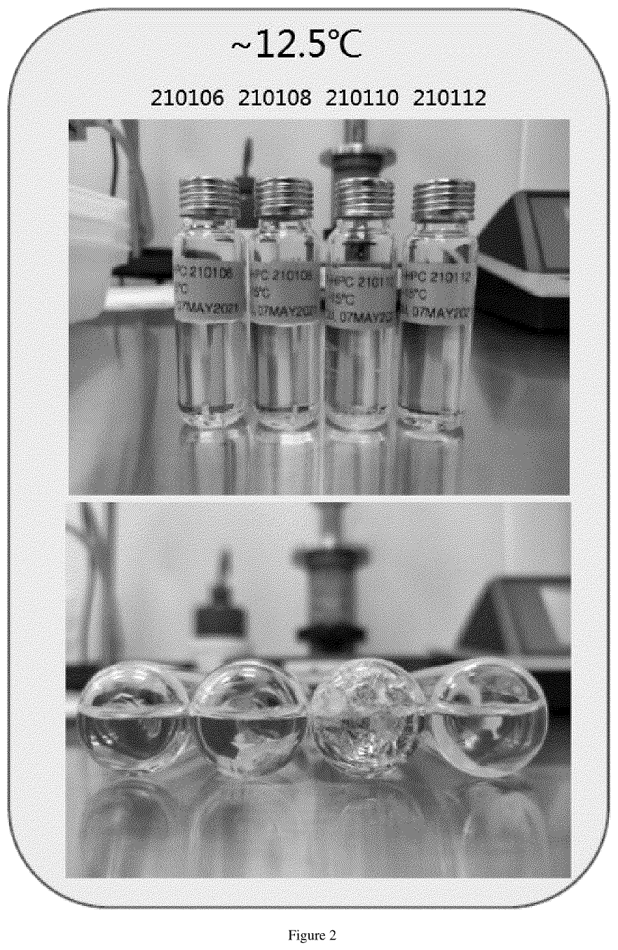

The physical stability test results under different conditions are shown in FIGS. 1-3:

At 5° C. for 72 hours: Only formulation 106 on the far left showed no precipitation; the remaining three formulations exhibited various degrees of precipitation.

At 12.5° C. for 72 hours: Similarly, only formulation 106 on the far left showed no precipitation; the other three formulations showed various degrees of precipitation.

At 20° C. for 72 hours: With the increase in temperature, the physical stability of all four formulations improved. Formulations 106 (the first from the left) and 110 (the third from the left) showed no precipitation.

### Example 4 Dissolving Experiment

TABLE 8
Experimental High BB/Benzyl Benzoate Formulation.

| Component | Concentration | | mg/Capsule |
|---|---|---|---|
| Hydroxyprogesterone caproate | 25% w/w (250 mg/g) | | 120 |
| Benzyl Benzoate | 67.5% | w/w | 324 |
| | 7.5% | w/w | 36 |
| Total | | | 480 |

The formulation is a previously developed formulation by the applicant, which demonstrates good physical and chemical stability. However, due to the relatively high content of benzyl benzoate, the relative safety of the formulation needs to be improved. Therefore, the low benzyl benzoate formulation included in this patent was developed. For the dissolution test below, a parallel comparison was conducted between the high benzyl benzoate formulation (Formula BB) and the low benzyl benzoate formulations (Formula 106 and Formula 112).

### Method

The dissolution apparatus used was the Pion MicroDiss Plus, commonly employed for formulation screening purposes. The experiment involved assessing dissolution rates over time. Under non-leaky sink conditions, at a constant temperature of 37° C., six 250 mL dissolution vessels were utilized at 250 rpm with a magnetic stirring device. Due to the milky dispersion, dissolution evaluation through UV spectroscopy (online analysis) was not applicable; instead, dissolution was tested using HPLC. Samples were divided into total samples and aqueous phase samples. For the comparative dissolution curves of different formulations, the same amount of API was introduced into the same volume of solvent: for example, 120 mg (480 mg of the BB formulation and 800 mg of the newly developed formulation) in 250 mL of solvent. Sampling time points were as follows: T5 minutes, T15 minutes, T30 minutes, (T45 minutes), T60 minutes, T120 minutes.

### FaSSIF (Fed State Simulated Intestinal Fluid)

The average value of each point in the dissolution curve was the average of two dissolution vessels tested. For full samples, 400 μL of solvent was sampled once and mixed with 600 μL of acetonitrile for HPLC analysis. For aqueous phase samples, 1000 μL of solvent sample was centrifuged at 14.8 MRPM for 20 minutes. Then, 400 μL of the upper aqueous phase was sampled and mixed with 600 μL of acetonitrile for HPLC analysis. The data is as follows:

TABLE 9
FaSSIF Data

% Dissolve

| | | |
|---|---|---|
| Sampling | Aqueous | |
| Time | Phase | Complete |
| (minute) | Sample | Sample |

Average of Formula 106

| | | |
|---|---|---|
| 0 | 0.00 | 0.00 |
| 5 | 18.33 | 112.79 |
| 15 | 20.78 | 105.71 |
| 30 | 23.43 | 106.33 |
| 45 | 39.84 | 105.85 |
| 60 | 38.80 | 109.64 |

Average of Formula 112

| | | |
|---|---|---|
| 0 | 0.00 | 0.00 |
| 5 | 10.18 | 154.18 |
| 15 | 18.33 | 134.17 |
| 30 | 14.36 | 107.45 |
| 45 | 23.79 | 117.33 |
| 60 | 18.55 | 109.54 |

Average of Formula BB

| | | |
|---|---|---|
| 0 | 0.00 | 0.00 |
| 5 | 4.46 | 78.61 |
| 15 | 4.44 | 79.63 |
| 30 | 4.33 | 93.15 |
| 45 | 4.42 | 79.41 |
| 60 | 4.38 | 92.00 |

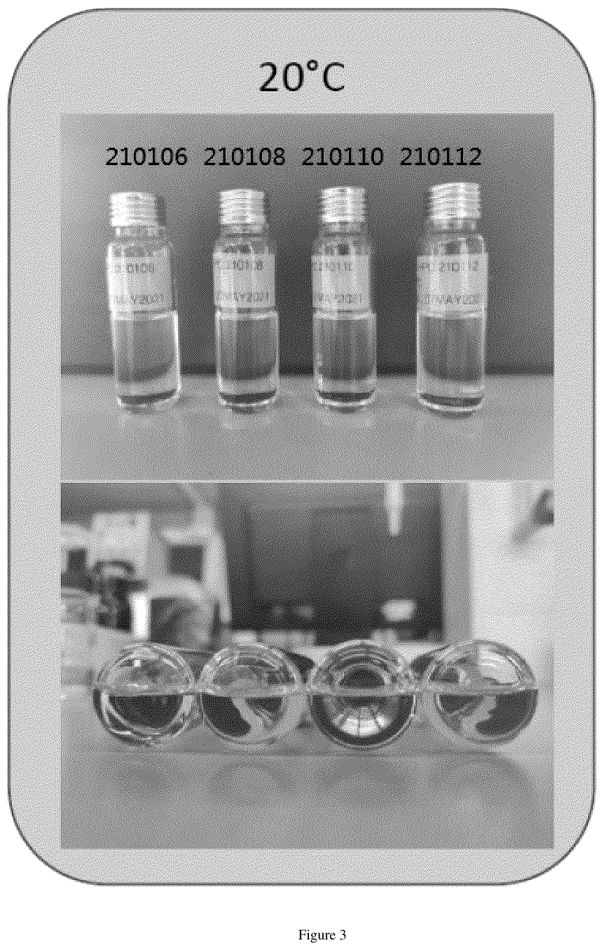

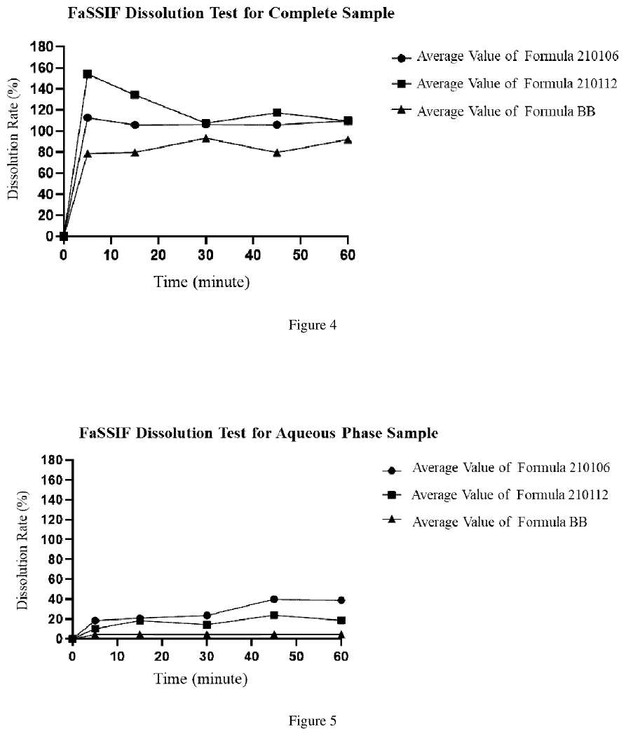

Comparison of 210106/210112/BB formulation in FaSSIF (Fasted State Simulated Intestinal Fluid) (FaSSIF dissolution medium, 37° C., 250 RPM, manual sampling) is shown in FIG. 4 (complete sample analysis) and FIG. 5 (water phase sample analysis, centrifugation at 14.8 MRPM for 20 minutes & sampling of supernatant). All three formulations reached close to 100% dissolution concentration within approximately 5 minutes. It should be noted that formulation 112 exceeded 100% dissolution concentration at 5 minutes because of its slower dispersion in the solvent, resulting in uneven sampling from the upper layer of the dissolution cup, which predominantly consisted of the oil phase, leading to higher concentrations. However, after some time of dissolution stirring, the concentration returned to around 100%. Moreover, based on the water phase dissolution curves, it was observed that both newly developed formulations exhibited higher dissolution concentrations compared to the original BB formulation, indicating higher API water solubility in these formulations, particularly in the prototype formulation 210106. Considering the experimental results and the results of physical stability experiments, the prototype formulation 210106 will be considered as the primary formulation for in vivo preclinical experiments.

### FaSSGF (Fasten State Simulated Gastric Fluid)

The average of each data point on the dissolution curve represents the average of two dissolution cup tests. For complete samples, 400 μL of solvent was sampled once and mixed with 600 μL of acetonitrile, followed by analysis using high-performance liquid chromatography (HPLC). Water phase samples were obtained by centrifuging 1000 μL of solvent at 14.8 MRPM for 20 minutes. Subsequently, 400 μL of the upper aqueous phase was collected and mixed with 600 μL of acetonitrile, followed by HPLC analysis. The data is presented below:

TABLE 10
FaSSGF Data

% Dissolve

| | | |
|---|---|---|
| Sampling | Aqueous | |
| Time | Phase | Complete |
| (minute) | Sample | Sample |

Average of Formula 106

| | | |
|---|---|---|
| 0 | 0.00 | 0.00 |
| 5 | 8.73 | 105.82 |
| 15 | 8.66 | 106.41 |
| 30 | 7.95 | 103.39 |
| 60 | 13.19 | 105.57 |
| 120 | 9.58 | 104.94 |

Average of Formula 112

| | | |
|---|---|---|
| 0 | 0.00 | 0.00 |
| 5 | 9.69 | 104.00 |
| 15 | 9.31 | 103.65 |
| 30 | 7.59 | 106.59 |
| 60 | 9.77 | 104.59 |
| 120 | 7.94 | 107.01 |

Average of Formula BB

| | | |
|---|---|---|
| 0 | 0.00 | 0.00 |
| 5 | 1.08 | 104.49 |
| 15 | 1.23 | 104.49 |
| 30 | 1.01 | 104.36 |
| 60 | 1.43 | 104.06 |
| 120 | 1.46 | 105.68 |

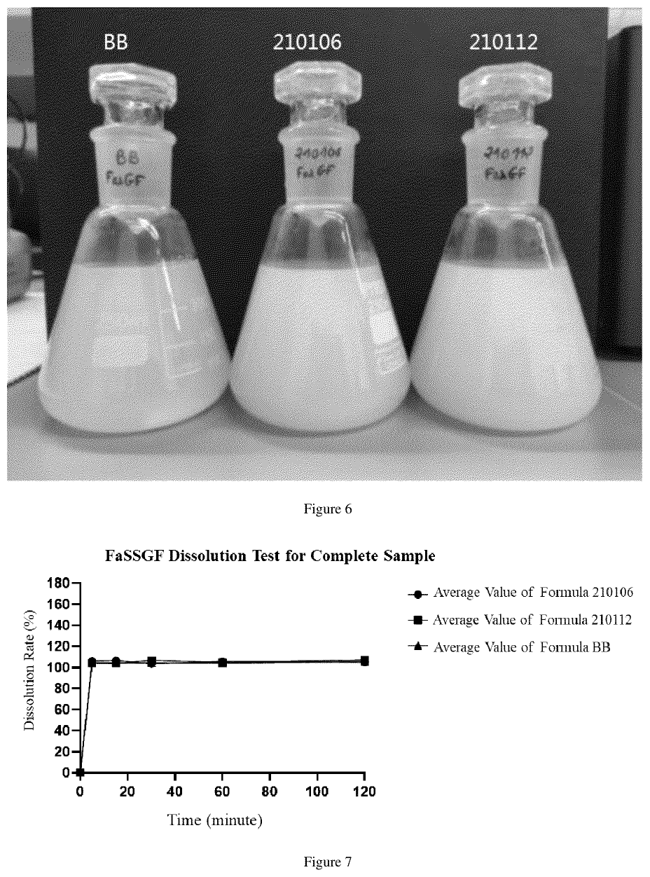

FIG. 6 shows the photograph of the solvent samples in FaSSGF (Fasted State Simulated Gastric Fluid) after stirring for 120 minutes. It can be observed that all three formulations appear as turbid suspensions, indicating that most of the API has not dissolved into the aqueous phase. This also explains the relatively low dissolution observed in the dissolution curves of the aqueous phase samples.

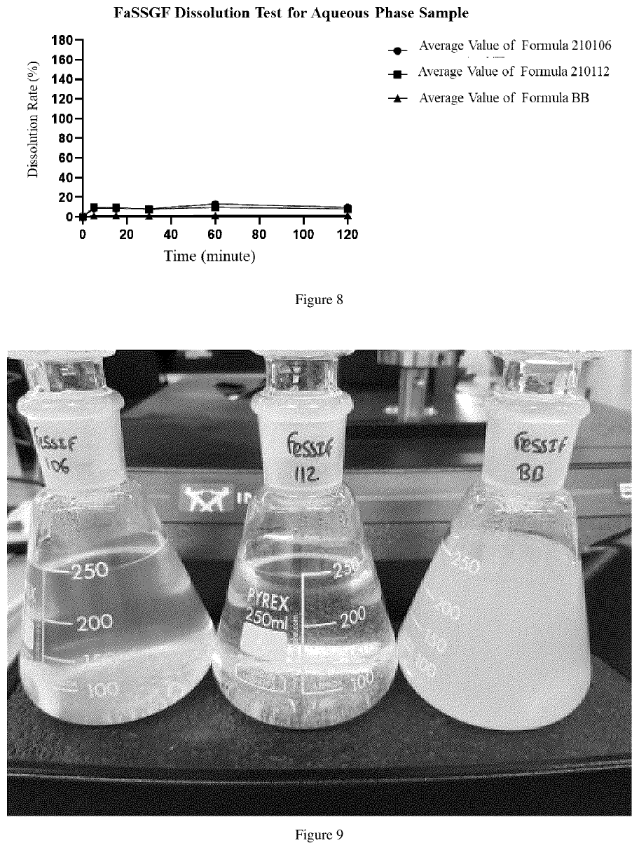

The comparison of the formulations 210106/210112/BB in FaSSGF (Fasten State Simulated Gastric Fluid) is presented in FIG. 7 (complete sample analysis) and FIG. 8 (aqueous phase sample analysis). The results indicate that all three formulations reach nearly 100% dissolution within approximately 5 minutes. However, under the acidic conditions simulating gastric fluid, only a small amount of API can dissolve into the aqueous phase. There is little difference observed among the three formulations under these gastric fluid dissolution conditions.

### FeSSIF (Fed State Simulated Intestinal Fluid)

The average value of each data point in the dissolution curve represents the average of two dissolution cup tests. Complete samples were obtained by taking a 400 μL solvent sample and adding 600 μL acetonitrile for subsequent analysis using high-performance liquid chromatography (HPLC). For the aqueous phase samples, a 1000 μL solvent sample was centrifuged at 14.8 MRPM for 20 minutes. Subsequently, 400 μL of the upper aqueous phase was extracted and mixed with 600 μL acetonitrile for HPLC analysis. The data is presented as follows:

TABLE 11
FeSSIF Data

% Dissolve

| | | |
|---|---|---|
| Sampling | Aqueous | |
| Time | Phase | Complete |
| (minute) | Sample | Sample |

Average of Formula 106

| | | |
|---|---|---|
| 0 | 0.00 | 0.00 |
| 5 | 52.41 | 102.28 |
| 15 | 51.54 | 105.82 |
| 30 | 53.44 | 104.33 |
| 60 | 54.44 | 106.22 |
| 120 | 46.37 | 101.20 |

Average of Formula 112

| | | |
|---|---|---|
| 0 | 0.00 | 0.00 |
| 5 | 58.71 | 103.31 |
| 15 | 60.44 | 106.23 |
| 30 | 61.31 | 104.96 |
| 60 | 46.43 | 101.01 |
| 120 | 43.78 | 104.00 |

Average of Formula BB

| | | |
|---|---|---|
| 0 | 0.00 | 0.00 |
| 5 | 17.07 | 105.52 |
| 15 | 15.39 | 106.18 |
| 30 | 15.88 | 106.37 |
| 60 | 14.87 | 105.95 |
| 120 | 15.10 | 106.62 |

FIG. 9 depicts the photograph of the solvent samples in FeSSGF (Fed State Simulated Gastric Fluid) at 120 minutes of dissolution agitation. It can be observed that in the gastric fluid simulated medium, formulations 106 and 112 exhibit much higher clarity compared to formulation BB, indicating that most of the API has dissolved into the aqueous phase, resulting in a lower turbidity. This also explains the phenomenon of higher dissolution rates of formulations 106 and 112 compared to formulation BB in the aqueous phase samples.

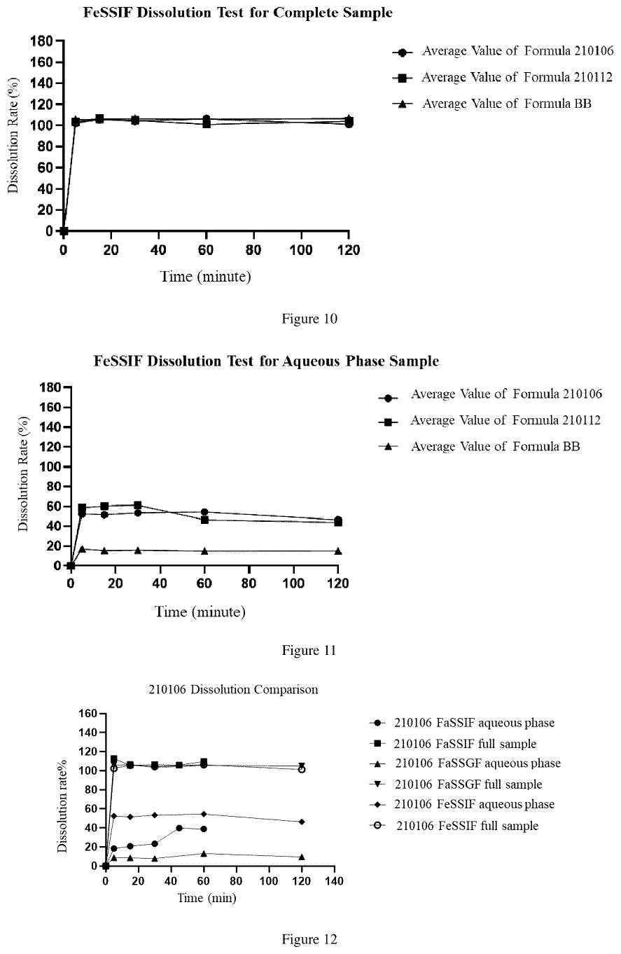

The comparison of formulations 210106/210112/BB in FeSSIF (Fed State Simulated Intestinal Fluid) is presented in FIG. 10 (complete sample analysis) and FIG. 11 (aqueous phase sample analysis). All three formulations reach close to 100% dissolution concentration in approximately 5 minutes. Moreover, according to the aqueous phase dissolution curves, it is observed that the two newly developed formulations exhibit higher dissolution concentrations in the aqueous phase compared to the original BB formulation. This advantage even exceeds the relative advantage of aqueous phase dissolution under FaSSIF (Fed State Simulated Intestinal Fluid) conditions. These dissolution curves demonstrate that the formulations exhibit higher API solubility, especially formulation prototype 210106. Based on the experimental results and physical stability test results, formulation prototype 210106 is considered as the primary formulation for preclinical in vivo experiments. Furthermore, these dissolution curves also suggest that the new formulations are likely to have higher bioavailability compared to the previous BB formulation, indicating that the API may be more effectively absorbed by the body upon ingestion of the new formulation compared to the original formulation.

The comparison of each candidate formulation in the three different dissolution media:

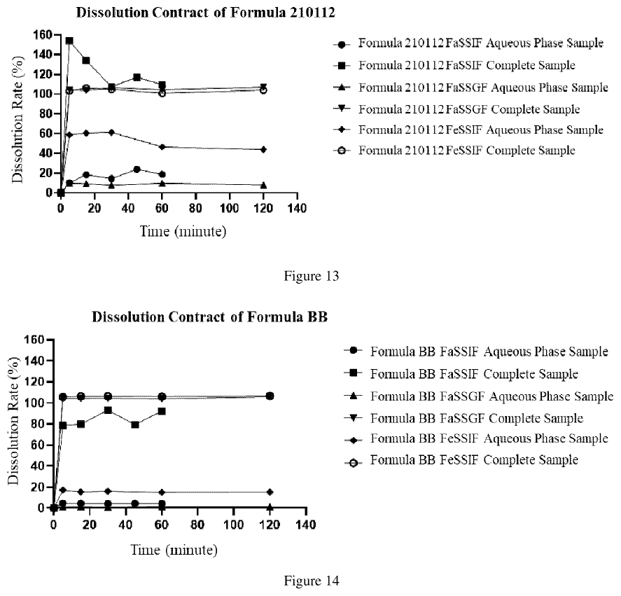

For each individual candidate formulation, we reanalyzed the same dataset to better understand and compare the dissolution behavior of each formulation in its corresponding three biorelevant media. The data is presented in Tables 12-14 and FIGS. 12-14.

TABLE 12
Dissolution Data of Formula 106

% Dissolve

| | | |
|---|---|---|
| Sampling | Aqueous | |
| Time | Phase | Complete |
| (minute) | Sample | Sample |

Average FaSSGF of Formula 106

| | | |
|---|---|---|
| 0 | 0.00 | 0.00 |
| 5 | 8.73 | 105.82 |
| 15 | 8.66 | 106.41 |
| 30 | 7.95 | 103.39 |
| 60 | 13.19 | 105.57 |
| 120 | 9.58 | 104.94 |

Average FeSSIF of Formula 106

| | | |
|---|---|---|
| 0 | 0.00 | 0.00 |
| 5 | 52.41 | 102.28 |
| 15 | 51.54 | 105.82 |
| 30 | 53.44 | 104.33 |
| 60 | 54.44 | 106.22 |
| 120 | 46.37 | 101.20 |

Average FaSSIF of Formula 106

| | | |
|---|---|---|
| 0 | 0.00 | 0.00 |
| 5 | 18.33 | 112.79 |
| 15 | 20.78 | 105.71 |
| 30 | 23.43 | 106.33 |
| 45 | 39.84 | 105.85 |
| 60 | 38.80 | 109.64 |

TABLE 13
Dissolution Data of Formula 112

% Dissolve

| | | |
|---|---|---|
| Sampling | Aqueous | |
| Time | Phase | Complete |
| (minute) | Sample | Sample |

Average FaSSGF of Formula 112

| | | |
|---|---|---|
| 0 | 0.00 | 0.00 |
| 5 | 9.69 | 104.00 |
| 15 | 9.31 | 103.65 |
| 30 | 7.59 | 106.59 |
| 60 | 9.77 | 104.59 |
| 120 | 7.94 | 107.01 |

Average FeSSIF of Formula 112

| | | |
|---|---|---|
| 0 | 0.00 | 0.00 |
| 5 | 58.71 | 103.31 |
| 15 | 60.44 | 106.23 |
| 30 | 61.31 | 104.96 |
| 60 | 46.43 | 101.01 |
| 120 | 43.78 | 104.00 |

Average FaSSIF of Formula 112

| | | |
|---|---|---|
| 0 | 0.00 | 0.00 |
| 5 | 10.18 | 154.18 |
| 15 | 18.33 | 134.17 |
| 30 | 14.36 | 107.45 |
| 45 | 23.79 | 117.33 |
| 60 | 18.55 | 109.54 |

TABLE 14
Dissolution Data of Formula BB

% Dissolve

| | | |
|---|---|---|
| Sampling | Aqueous | |
| Time | Phase | Complete |
| (minute) | Sample | Sample |

Average FaSSGF of Formula BB

| | | |
|---|---|---|
| 0 | 0.00 | 0.00 |
| 5 | 1.50 | 104.49 |
| 15 | 1.23 | 104.49 |
| 30 | 1.01 | 104.36 |
| 60 | 1.43 | 104.06 |
| 120 | 1.46 | 105.68 |

Average FeSSIF of Formula BB

| | | |
|---|---|---|
| 0 | 0.00 | 0.00 |
| 5 | 17.07 | 105.52 |
| 15 | 15.39 | 106.18 |
| 30 | 15.88 | 106.37 |
| 60 | 14.87 | 105.95 |
| 120 | 15.10 | 106.62 |

Average FaSSIF of Formula BB

| | | |
|---|---|---|
| 0 | 0.00 | 0.00 |
| 5 | 4.46 | 78.61 |
| 15 | 4.44 | 79.63 |
| 30 | 4.33 | 93.15 |
| 45 | 4.42 | 79.41 |
| 60 | 4.38 | 92.00 |

The dissolution performance comparison of the three formulations in the three media FaSSIF (fasted state simulated intestinal fluid), FeSSIF (fed state simulated intestinal fluid), and FaSSGF (fasted state simulated gastric fluid) is illustrated in FIGS. 12-14. By comparing the dissolution media for the same formulation type, we observed the following phenomenon: solubility in aqueous phase: FeSSIF (fed state intestinal fluid)>FaSSIF (fasted state intestinal fluid)>FaSSGF (fasted state gastric fluid). This is likely due to the higher pH in intestinal fluid compared to gastric fluid, as well as the higher concentration of bile salts in intestinal fluid compared to gastric fluid. Additionally, the surfactant excipients added to the new formulation may have contributed to increased solubility in water, facilitating better absorption of the API in the aqueous phase, thus potentially improving the bioavailability of the new formulation.

This application aims to enhance the chemical stability of low benzyl benzoate oral formulations, thereby expanding their clinical and commercial utility. Improved chemical stability not only reduces production costs but also lowers storage and transportation expenses, thereby increasing the economic and commercial value of the product.

This application enhances the bioavailability of low-benzoate oral formulations as proposed in this application can reduce the dosage required, thereby improving the practicality of drug administration for patients. Experimental results indicate that in experiments simulating non-fasted state intestinal fluid, the dissolution rate of the aqueous phase for the low benzyl benzoate oral formulation is over 8.8 times higher compared to the original high benzyl benzoate oral formulation. This suggests that the bioavailability of the low benzyl benzoate oral formulation is over 8.8 times higher than that of the original high benzyl benzoate oral formulation, potentially allowing for a dosage reduction of up to 8.8 times compared to the original high benzyl benzoate formulation. Consequently, this significantly enhances the clinical and commercial application value of the low benzyl benzoate formulation.

This application also significantly enhances the safety of the low-benzoate formulation compared to the original high benzyl benzoate formulation. The excipients used in the low benzyl benzoate formulation comply with safety requirements, and the daily dosage of excipients also meets safety standards and requirements. This substantially increases the applicability and commercial value of the low benzyl benzoate formulation.

1. A novel oral formulation containing reproductive hormones, characterized in that the oral formulation does not contain or contains only low levels of benzyl benzoate.

2. The oral formulation according to claim 1, wherein the reproductive hormone is 17-α hydroxyprogesterone caproate.

3. The oral formulation according to claim 1, wherein the weight percentage of benzyl benzoate in the oral formulation is less than 8%; preferably, less than 7%; more preferably, less than 6%; even more preferably, less than 5%.

4. The oral formulation according to claim 1, wherein the weight percentage of benzyl benzoate in the oral formulation is less than 4%; preferably, less than 3%; more preferably, less than 2%; even more preferably, less than 1%.

5. (canceled)

6. The oral formulation according to claim 1, wherein the oral formulation includes propylene glycol monocaprylate.

7. The oral formulation according to claim 1, wherein the oral formulation includes 40-60% by weight, preferably 45-55%, more preferably 50-52%, even more preferably 51.8%-51.9% of propylene glycol monocaprylate, most preferably 51.85% of propylene glycol monocaprylate.

8. The oral formulation according to claim 1, wherein the oral formulation includes PEG35 castor oil.

9. The oral formulation according to claim 1, wherein the oral formulation includes 5-10% by weight, preferably 6-9%, more preferably 7-8%, even more preferably 7.6%-7.7% of PEG35 castor oil.

10. The oral formulation according to claim 1, wherein the weight percentage of reproductive hormone in the oral formulation is 10-20%; preferably, 12-18%; more preferably, 15%.

11. The oral formulation according to claim 1, wherein the oral formulation includes propylene glycol monolaurate.

12. The oral formulation according to claim 11, wherein the oral formulation includes 15-30% by weight, preferably 20-25% of propylene glycol monolaurate, more preferably 20.8%-20.9% of propylene glycol monolaurate, most preferably 20.825% of propylene glycol monolaurate.

13. The oral formulation according to claim 1, wherein the oral formulation includes glycerol monocaprylate-, preferably the oral formulation includes 5-25% by weight, preferably 8-22% of glycerol monocaprylate.

14. (canceled)

15. The oral formulation according to claim 1, wherein the oral formulation includes 17-α hydroxyprogesterone caproate, propylene glycol monocaprylate, PEG35 castor oil, benzyl benzoate, and propylene glycol monolaurate or glycerol monocaprylate, preferably the oral formulation includes 10-20% by weight of 17-α hydroxyprogesterone caproate, 45-55% of propylene glycol monocaprylate, 5-10% of PEG35 castor oil, 1-6% of benzyl benzoate, and 20-25% of propylene glycol monolaurate or 9-22% of glycerol monocaprylate.

16. (canceled)

17. The oral formulation according to claim 15, wherein the oral formulation consists of 15% by weight of 17-α hydroxyprogesterone caproate, 51.85% of propylene glycol monocaprylate, 7.65% of PEG35 castor oil, 4.675% of benzyl benzoate, and 20.825% of propylene glycol monolaurate.

18. The oral formulation according to claim 1, wherein the oral formulation includes 17-α hydroxyprogesterone caproate, propylene glycol monocaprylate, PEG35 castor oil, and propylene glycol monolaurate, preferably, the oral formulation consists of 10-20% by weight of 17-α hydroxyprogesterone caproate, 45-55% of propylene glycol monocaprylate, 5-10% of PEG35 castor oil, and 20-30% of propylene glycol monolaurate.

19. (canceled)

20. The oral formulation according to claim 18, wherein the oral formulation consists of 15% by weight of 17-α hydroxyprogesterone caproate, 51.85% of propylene glycol monocaprylate, 7.65% of PEG35 castor oil, and 25.50% of propylene glycol monolaurate.

21. The oral formulation according to claim 1, wherein the oral formulation is a commonly used medical dosage form, including oral liquid or soft capsule formulations, with a preference for the soft capsule form.

22. The oral formulation according to claim 1, wherein each unit dose contains 80-150 mg of reproductive hormones, preferably 100-120 mg.

23-24. (canceled)

25. A method for contraception, treating miscarriage, menstrual irregularities, uterine bleeding, breast cancer, cytokine storm syndrome, or COVID-19, characterized by the step of using the oral formulation according to claim 1.

26. A method for preparing the oral formulation according to claim 1, comprising the steps of weighing excipients, pre-dissolving excipients, mixing excipients, adding reproductive hormones, and dissolving reproductive hormones.
